(19) United States
(12) Patent Application Publication
(10) Pub. No.: US 2013/0116244 A1
(43) Pub. Date: May 9, 2013

(54) PHARMACEUTICAL POWDER COMPOSITIONS

(71) Applicants: James Coghill, Nottingham (GB); Robert Armstrong, Nottingham (GB)

(72) Inventors: James Coghill, Nottingham (GB); Robert Armstrong, Nottingham (GB)

(73) Assignee: AESICA FORMULATION DEVELOPMENT LIMITED, Nottingham (UK)

(21) Appl. No.: 13/695,113

(22) PCT Filed: Apr. 28, 2011
(86) PCT No.: PCT/GB11/00671

§ 371 Date: Jan. 9, 2013

(30) Foreign Application Priority Data
Apr. 30, 2010 (GB) 1007290.8

(51) Int. Cl. A61K 9/00

(52) U.S. Cl. CPC A61K 9/006
USPC 514/221

(57) ABSTRACT

The present invention provides a powder composition for the buccal delivery comprising a drug compound, a muco-adhesive agent and a dispersing agent Suitable compounds drugs include benzodiazepine compounds. Suitable muco-adhesive agents include those selected from (t) esters and salts of alginic acid and (it) hydroxyethyl cellulose. Suitable dispersing agents include those selected from ethylene oxide-propylene oxide copolymers.

### FIELD OF THE INVENTION

This invention relates to pharmaceutical compositions for the buccal administration of drug compounds, such as benzodiazepine drug compounds.

### BACKGROUND OF THE INVENTION

The buccal route of drug delivery can afford rapid absorption of drugs into the blood circulation. The buccal route, i.e. administration through the buccal mucosa, mainly composed of the lining of the cheeks, can also offer a less invasive route of drug administration compared with some other routes such intravenous or intramuscular injection. Such rapid and effective drug delivery can be useful in the treatment of crisis situations such as pain (including breakthrough pain, headache), migraine, anxiety, status epilepticus (epilepsy), convulsions, impotence and nausea.

A class of compounds of interest for buccal delivery is the benzodiazepines.

These lipophilic drugs act on the central nervous system to cause sedation, hypnosis, decreased anxiety, muscle relaxation, anterograde amnesia and anticonvulsant actions and are widely used in medicine. Conditions which they can be used to treat include anxiety, epilepsy, insomnia, alcohol dependence, muscular disorders and mania. These drugs can also be used in premedication procedures and in veterinary practices.

Examples of benzodiazepine drugs include but are not limited to alprazolam, chlordiazepoxide, clonazepam, clorazepate, diazepam, estazolam, flurazepam, halazepam, lorazepam, midazolam, nitrazepam, oxazepam, prazepam, quazepam, temazapem, bromazepam, flunitrazepam, triazolam, bentazepam, brotizolam, clotiazepam, delorazepam, ethyl loflazepate, etizolam, fludiazepam, ketozolam, loprazolam, lormetazepam, nordazepam, mexazolam, nimetazepam, pinazepam and tetrazepam. The structures of some of these benzodiazepines can be found in Goodman and Gilman's The Pharmacological Basis of Therapeutics, 9th edition, McGraw Hill (1996), page 363.

The listing or discussion of an apparently prior-published document in this specification should not necessarily be taken as an acknowledgement that the document is part of the state of the art or is common general knowledge.

A preferred means of delivering drugs for systemic action by the buccal route is using an aqueous solution formulation. However, benzodiazepines typically have low aqueous solubility and hence present a significant challenge when designing an aqueous formulation for buccal delivery.

One benzodiazepine that presents particular challenges is lorazepam. Lorazepam has high-potency and is an intermediate-acting benzodiazepine. Its properties are largely explained by its pharmacokinetic properties (poor water and lipid solubility, high protein binding and non-oxidative metabolism to a pharmacologically inactive glucuronide form) and by its high relative potency (lorazepam 1 mg is equal in effect to diazepam 10 mg). The half life of lorazepam is 10-20 hours.

Because of its poor lipid solubility lorazepam is absorbed relatively slowly by mouth and is unsuitable for rectal administration. But its poor lipid solubility and high degree of protein binding (85-90%) mean that lorazepam's volume of distribution is mainly in the vascular compartment, causing relatively prolonged peak effects. This contrasts with the highly lipid-soluble diazepam, which, although rapidly absorbed orally or rectally, soon redistributes from the serum to other parts of the body, in particular body fat. Thus, one lorazepam dose, despite lorazepam's shorter serum half-life, can have more prolonged peak effects than an equivalent diazepam dose. On regular administration diazepam may accumulate more, since it has a longer half-life and active metabolites with even longer half-lives.

Diazepam has long been a drug of choice for status epilepticus. Its high lipid solubility means it is absorbed with equal speed whether given intravenously, orally, or rectally. However, diazepam's high lipid solubility also means it does not remain in the vascular space but soon redistributes into other body tissues. Therefore, it may be necessary to repeat diazepam doses to maintain peak anticonvulsant effects, this can result in excess body accumulation.

Lorazepam, on the other hand has low lipid solubility so that it is relatively slowly absorbed when administered by any route other than intravenous injection, but once administered will not get significantly redistributed beyond the vascular space. Therefore, lorazepam's anticonvulsant effects can be more durable, thus potentially reducing the need for repeated doses. If a patient is known to usually stop convulsing after only one or two diazepam doses, diazepam may be preferable because sedative after-effects will be less than if a single dose of lorazepam is given (diazepam anticonvulsant/sedative effects typically wear off after 15-30 minutes, but lorazepam effects can last for 12 to 24 hours).

Some benzodiazepines, such as lorazepam, are unstable in liquid formulations. Even when stored at 4 to 8° C., liquid formulations comprising lorazepam have a limited shelf-life.

One lorazepam formulation for intramuscular injections comprises polyethylene glycol (about 18%), propylene glycol (>about 78%), benzyl alcohol (about 2%) and the drug. To produce such a solution, lorazepam is dissolved in polyethylene glycol and the resulting solution is diluted with propylene glycol. Typically the polyethylene glycol and propylene glycol both have a molecular weight of around 400. Benzyl alcohol is used as a preservative. Benzyl alcohol is contraindicated in infants and children up to 3 years old. It would therefore be advantageous to provide benzodiazepine containing formulations, such as lorazepam containing formulations, which do not comprises benzyl alcohol.

Some benzodiazepines, such as lorazepam, are not stable when subjected to standard heat sterilisation procedures known in the art. Compositions containing such benzodiazepines should be prepared aseptically.

### SUMMARY OF THE INVENTION

The present inventors have found that lipophilic and/or poorly water soluble drugs, such as benzodiazepines, can be successfully administered buccally, if delivered in the form of a powder as described below. By successfully administered, we mean that therapeutically effective amounts of drug can be absorbed into the systemic circulation of a patient, e.g. a human.

By the term “poorly water soluble” we mean that the drug has a solubility in water at 20° C. of not more than about 1 mg/ml. Such drugs are sometimes referred to in the literature as “very slightly soluble” (solubility in water at 20° C. of from 0.1 to 1 mg/ml) and “practically insoluble” or “insoluble” (for both, solubility in water at 20° C. of less than 0.1 mg/ml).

The present invention provides a powder composition for buccal delivery comprising a benzodiazepine drug compound; a muco-adhesive and a dispersing agent.

Any suitable muco-adhesive may be used. Muco-adhesives are materials that adhere to the mucus. Suitable muco-adhesives include those selected from (i) esters and salts of alginic acid; and (ii) hydroxyethyl cellulose; and mixtures thereof.

Alginic acid has the general formula (C₆H₈O₆)ₙ and has a molecular weight of from 10,000 to 600,000.

Suitable salts of alginic acid for use in the present invention include alkali metal and alkaline earth metal salts, such as, sodium alginate (empirical formula NaC₆H₇O₆), the potassium salt of alginic acid and the calcium salt of alginic acid; and combinations thereof.

Suitable esters of alginic acid include alkyl diol esters, such as, propylene glycol alginate.

Hydroxyethyl cellulose may be used as the muco-adhesive agent. Suitable hydroxyethyl cellulose materials include those sold under the trade name Natrosol®, such as, Natrosol® 250 HX, available commercially in the UK.

By way of example, other materials that may have muco-adhesive properties and that may optionally be included in the buccal delivery powder composition of the present invention include, but shall not be limited to, C₁-C₈ alkyl vinyl ether/organic acid copolymers, such as, copolymers of a methyl or ethyl vinyl ether with an acid, such as, malic acid, for example, Gantrez® which is a methylvinylether/malic acid copolymer (PVM/MA copolymer).

The muco-adhesive is typically present in the composition of the present invention in an amount of from 0.1 to 90% w/w, preferably from 1 to 60% w/w, preferably from 5 to 40% w/w, preferably from 20 to 30% w/w, more preferably from 25 to 29% w/w.

The muco-adhesives used in the composition of the present invention typically have a viscosity of from about 500 to about 4000 mpa, for example from about 700 to about 2500 mpa. For example, propylene glycol alginate has a viscosity of from about 700 to about 1800 mpa in 2% aqueous solution and Natrosol® 250 HX has a viscosity from about 1500 to 2500 mpa in 1% aqueous solution.

The compositions of the invention comprise at least one dispersing agent. Typically, the dispersing agent is a hydrophilic/lipophilic copolymer. Suitable dispersing agents include but are not limited to ethylene oxide-propylene oxide copolymers, often known as poloxamers, for example poloxamer 188 and other poloxamers. Other materials suitable for use as dispersing agents will be within the knowledge of the skilled person.

The primary purpose of a dispersing agent is to facilitate dispersion of the drug on administration. The dispersing agent may enhance the bioavailability of the active ingredient and/or may act as a water soluble lubricant. Suitable dispersing agents include non-ionic copolymers with a hydrophilic segment and a hydrophobic segment such materials can have emulsifying and solubilising properties.

Poloxamer 188 is particularly suitable for use as a dispersing agent in the present invention. It is believed that, as well as being a dispersing agent, poloxamers may provide additional benefits. Poloxamers and especially poloxamer 188 may potentially act as a solubilising agent and/or absorption enhancer. Poloxamers and especially poloxamer 188 also has the potential to act as a taste-making agent and/or contribute to a pleasant mouth feel. Additionally, poloxamers and especially poloxamer 188 may provide advantageous lubricant properties in automated filing operations.

Other suitable dispersing agents include polyvinyl caprolactam-polyvinyl acetate-polyethylene glycol graft copolymers such as those sold under the name Soluplus. It will be appreciated that dispersing agents other than poloxamer 188 may be used.

The dispersing agent is typically present in the composition of the present invention in an amount of from 1 to 50% w/w, preferably from 10 to 40% w/w, more preferably from 15 to 25% w/w.

This invention can be applied to any benzodiazepine compound. Examples of benzodiazepine compounds include, but are not limited to, those selected from the group alprazolam, chlordiazepoxide, clonazepam, clorazepate, diazepam, estazolam, flurazepam, halazepam, lorazepam, midazolam, nitrazepam, oxazepam, prazepam, quazepam, temazapem, bromazepam, flunitrazepam, triazolam, bentazepam, brotizolam, clotiazepam, delorazepam, ethyl loflazepate, etizolam, fludiazepam, ketozolam, loprazolam, lormetazepam, nordazepam, mexazolam, nimetazepam, pinazepam and tetrazepam, and optionally combinations thereof.

Other suitable drugs include but are not limited to other drugs that could be administered via buccal or sub-lingual routes such as anti-malarial drugs, galantamine, neostigmine, physostigmine, edrophoniumlevobupivacaine, lidocaine, prilocaine, mepivacaine, propofol, rapacuronium bromide, ropivacaine, tubocurarine, atracurium, doxaurium, mivacurium, pancuronium, vercuronium, pipecuronium, rocurronium amantadine, ipratropium, oxitropium, dicycloverineacetazolamide, carbamazepine, divalproex, ethosuximide, lamotrignine acid, levetriacetam, oxcarbazepine, Phenobarbital, phenyloin, pregabalin, primidone, remacemide, trimethadione, topiramate, vigabatrin, zonisamideamisulpride, aripiprazole bifemelane, bromperidol, clozapine, chlorpromazine, haloperidol, iloperidone loperidone, olanzapine, quetiapine, fluphenazine, esperidone, thiothixene, thioridazine, sulpride, ziprasidone amitryptiline, atracurium, buspirone, chlorzoxazone, clorazepate, cisatracurium, cyclobenzaprine, eperisone, esopiclone, hydroxyzine, mirtazapine, mivacurium, pagoclone, superide, zaleplon, zopicloneentacapone, lazebemide, selegiline, tolcaponeglatiramer, estradiol, proqesteronememanine, nimodipine, riluzole, rivastigmine, tacrine, xaliprodencarbidopa, levodopa, tacrine, donezepil, rivastigmine, galantamiacetylcholine, serotonin, 5-hydroxytryptamine (5-HT), GABA, glutamate, aspartate, glycine, histamine, epinephrine, norpinephrine, dopamine, adenosine, ATP nitric oxidealmotriptan, aniracetam, atomoxetine, benserazide, bromocriptine, bupropion, cabergoline, citalopram, clomipramine, desipramine, dihydroergotmine, doxepin duloxetine, eletriptan, escitalopram, fluvoxamine, gabapentin, imipramine, moclobemide, naratriptan, nefazodone, nefiracetam acamprosate, nicergoline, buprenorphine, naloxone, apomorphine.

A preferred benzodiazepine drug for use in this invention is lorazepam (7-chloro-5-(2-chlorophenyl)-1,3-dihydro-3-hydroxy-2H-1,4-benzodiazepin-2-one).

Methods of formulating drug substances for administration in a powder form are well known to those skilled in the art. Any such method may be used to formulate the composition of the present invention. For example, the benzodiazepine drug may be formulated as a blend of drug powder with other ingredients, as granules or microspheres or as a freeze-dried powder. The term powder used herein shall include any fine, loose, substantially free flowing particles, including, for example, dry powders, granules, pellets, microspheres, and the like.

The simplest form of the powder compositions of the invention is a blend of the drug, such as a benzodiazepine drug and the muco-adhesive agent, the dispersing agent and any other ingredients.

The compositions of the invention may optionally comprise one or more additional ingredients that are standard in the art. Such ingredients include but are not limited to:

Sweeteners such as sugar alcohols such as xylitol, mannitol, isomalt and sorbitol and mixtures of sugar alcohols or dextrates or other saccharides;
disintegrants such as crospovidone, croscarmellose sodium, sodium alginate, and sodium starch glycollate
solubilising agents such as polyethylene glycol, for example polyethylene glycols having a molecular weight of from about 8,000 to about 35,000, such as Polyethylene Glycol 20000;
glidants such as colloidal silica, talc, magnesium trisilicate, powdered cellulose, tribasic calcium phosphate and starch;
lubricants such as magnesium stearate, sodium stearyl fumarate and hydrogenated vegetable oil; surfactants such as polysorbates, docusate sodium, sodium lauryl sulphate, sorbitan esters;
additional sweeteners such as Acesulfame potassium, aspartame and saccharine; flavourants such as menthol or mint flavourants, for example Novomint Freshmint 506038 TP0504.

The sweeteners such as sugar alcohols used in the present invention may act as carriers and/or blending aids and/or sweeteners. It is particularly preferred to use xylitol. isomalt and/or mannitol. The use of xylitol is well known to be advantageous to dental health.

If a sugar alcohol is present in a composition of the present invention it is typically present in an amount of from 0.1 to 90% w/w. preferably from 5 to 70% w/w, more preferably from 10 to 50% w/w.

The solubilising agents used in the present invention are typically water soluble and have the potential to increase the solubility and absorption of the benzodiazepine drug. Some solubilising agents may also act as lubricants in automated filing operations. Polyethylene glycol is particularly suitable for use as a solubilising agent in the present invention, although other materials with similar properties may alternatively or additionally be used.

If a solubilising agent is present in a composition of the present invention it is typically present in an amount of from 0.1 to 20% w/w, preferably from 1 to 10% w/w, more preferably from 5 to 8% w/w.

It is desirable for the compositions of the invention to have a pleasant mouth feel following administration. Compositions that have a pleasant mouth feel include those that form a smooth gel in the mouth (generally, a smooth gel has no grittiness). The flavour can also contribute to mouth feel. Mint and/or menthol flavours can contribute to providing a pleasant mouth feel.

Preferred disintegrants for use in the present invention also have the potential to enhance the solubility of the drug compound. Suitable disintergrants include crospovidones such as Polyplasdone XL 10. Polyplasdone XL 10 has excellent wicking properties which can help prevention of particle agglomeration, whilst also providing smooth mouth feel. Polyplasdone XL 10 may also enhance dissolution of poorly soluble actives. Other suitable disintegrants are known in the art.

If a disintegrant is present in a composition of the present invention it is typically present in an amount of from 0.1 to 10% w/w, preferably from 1 to 5% w/w.

The flavourants that are preferably used in the present invention impart a pleasant flavour and mask the taste of ingredients with an unpleasant taste, such as the drug compound. Some flavourants may also improve the mouth feel of the compositions of the invention and/or act as an absorption enhancer. One flavourant that has this combination of properties is Novamint Freshmint 506038 TP0504. It will be appreciated that other flavourants known in the art may alternatively or additionally be used.

If a flavourant is present in a composition, of the present invention it is typically present in an amount of from 0.1 to 10% w/w, preferably from 1 to 5% w/w.

For preparing a substantially uniform powder blend on a small scale, a pestle and mortar and/or sieve may be appropriate whereas mechanical mixers are required for larger scale manufacture. There are numerous types of mixer available and these are widely described in the literature, for example Chapter 37, Remington: The Science and Practice of Pharmacy, 20th Edition, Lipincott, Williams and Wilkins, Baltimore, 2000. By “substantially uniform” we mean the drug is evenly distributed within the formulation such that if the theoretical weight of powder in which the drug dose is contained is analysed for drug content the result will be in the range 80-120% of the nominal amount i.e. if 5 mg of drug is theoretically contained within 10 mg of powder formulation, the drug distribution will be considered substantially uniform if the assayed amount of drug in 10 mg of powder is in the range 4-6 mg.

Although the formation of a powder blend is the simplest approach, there are a number of other methods that may be used, For example, methods such as granulation, microencapsulation and lyophilisation can be used and are particularly useful when there is a need to control particle size and for uniformity of drug distribution within the composition. Other suitable methods of formulation include spray drying and super critical fluid processes. Of these other methods, granulation is preferred since it is of relatively low complexity and an economical process.

In one aspect, the compositions of the invention comprise granules of one or more ingredients of the composition. A combination of ingredients, such as the drug and the muco-adhesive agent, and optionally other ingredients hereinbefore described, may be incorporated within a single granule. Granules are agglomerates of smaller particles and may be produced by techniques well known to those skilled in the art such as wet granulation, dry granulation (slugging), extrusion/spheronisation, fluid bed granulation and spray congealing. In addition to the drug and the muco-adhesive agent, other ingredients incorporated into the granules may include the additional ingredients described above such, as a sugar alcohol, a dispersing agent, a disintegrant, a solubilising agent; a glidant, a lubricant, a sweetener, and/or a flavourant.

Granules may include binders such as povidone (polyvinylpyrrolidone), methylcellulose, polyethylene glycol, gelatin and acacia and alternative or additional disintegrants such as starch, croscarmellose and crospovidone.

Dry granulation techniques are particularly suitable for preparing compositions comprising lorazepam or other drugs sensitive to water or other solvents. Suitable dry granulation techniques are well known in the art and include roller compaction.

In a wet granulation process, a solution of a binding agent in an aqueous or organic solvent (the granulating solvent) is mixed into the solid ingredients (e.g. drug) to form a homogeneous mass. The mass is passed through a coarse mesh and the aqueous or organic solvent removed by evaporation/drying to produce granules. The granules may then, if required, be further milled and sieved to produce particles of the desired size.

The binding agent is typically dissolved in the granulating solvent, alternatively it may be added in dry form to the powder mixture and the solvent added to the powder blend to form the homogeneous mass. Further details on the process of granulation may be found in the literature, for example Chapter 6, Pharmaceutical Principles of Solid Dosage Forms, J. T. Carstensen, Technomic, Lancaster, Pa. 1993.

It is preferable that benzodiazepine drug and the muco-adhesive are contained within the same granule, however, other configurations are possible. Examples of other configurations include a blend of (non-granule form) and drug granules, a blend of granules comprising the muco-adhesive and benzodiazepine drug containing granules; and a blend of benzodiazepine drug powder (non-granule form) and granules comprising the muco-adhesive. In all cases other ingredients, as described earlier, may be added as necessary to the granule and/or non-granule component of the composition.

A substantially uniform powder composition may also be prepared by a process involving lyophilisation (freeze-drying). A solution or suspension of drug and the other ingredients of the composition is prepared, including additional ingredients as required. The solution is frozen and lyophilised to leave either a powder as a plug or in a dispersed (free-flowing) form. The particle size of powders produced by freeze drying is sometimes heterogeneous and poorly defined. It may, therefore, be necessary for the lyophilised powder to undergo a process to produce particles of a well defined size. Methods for reduction of particle size, which may also be applied to the granule compositions, are well known in the art. One method for reducing the particle size of the compositions is milling and/or sieving. There are several types of mill available and these are widely described in literature references, see for example Chapter 2, Pharmaceutical Principles of Solid Dosage Forms, J. T. Carstensen, Technomic, Lancaster, Pa., 1993 and Chapter 37, Remington: The Science and Practice of Pharmacy, 20th Edition, Lipincott, Williams and Wilkins, Baltimore, 2000 The compositions of the invention may be in the form of microspheres. Methods for preparation of microspheres are well known in the art and include, but are not limited to, spray drying, interfacial polymerisation, coarcervation/phase separation and solvent evaporation. Methods for producing microspheres are described in, for example, Physicochemical Principles of Pharmacy, 3rd Edition, pages 357 to 360, A T Florence and D Attwood, Macmillan, London, 1998 and Physical Pharmacy, 4^{th} Edition, pages 516 to 519, A Martin, Wilkins and Wilkins, Baltimore, 1993.

In addition to a benzodiazepine drug compound and the muco-adhesive and the additional ingredients hereinbefore described, the microspheres may include ingredients that are known in the art to be suitable to be included in microspheres. Such ingredients include, but are not limited to, starches, dextrans, gelatin, albumin, collagen, hyaluronic acid, lactose, sucrose, dextrose, mannitol, methacrylate copolymers such as the Eudragit® polymers (Degussa, Germany), celluloses, such as, methylcellulose, and polyesters such as poly(lactide-co-glycolide).

The powder compositions of the present invention preferably have a benzodiazepine drug content of from about 0.1 to about 50% w/w of the composition, more preferably from about 1 to about 20% w/w and most preferably from about 2 to about 10% w/w, for example 3, 4, 5, 6, 7, 8 or 9% w/w. The remainder of the powder composition comprises other ingredients, as hereinbefore described.

When additional ingredients are included in the composition of the invention, they may simply be added to the compositions comprising the drug and the muco-adhesive in an amount as defined above. Alternatively, they may replace a portion of the muco-adhesive.

A preferred composition of the invention comprises a benzodiazepine (for example lorazepam) a muco-adhesive such as propylene glycol alginate and optionally a sugar alcohol such as xylitol. Preferred proportions of these three components are provided in Table I below:

Table I

| | Composition (% w/w) | | |
|---|---|---|---|
| | Preferred | More preferred | Most preferred |
| Benzodiazepine | 0.1-80 | 0.5-50 | 1-20 |
| Muco-adhesive | 0.1-90 | 1-60 | 5-40 |
| Dispersing agent | 1-50 | 10-40 | 15-25 |
| Sugar alcohol | 1-90 | 5-70 | 10-50 |

An example of a preferred lorazepam-containing composition of the invention comprises from 3 to 10% w/w, for example 4 to 6% w/w, lorazepam; from 20 to 30% w/w, for example 25 to 29% w/w of a muco-adhesive, such as propylene glycol alginate. Such a composition may optionally contain one or more of from 30 to 40% w/w, for example from 35 to 39% w/w, of a sugar alcohol, such as xylitol; from 10 to 40% w/w, for example from 15 to 25% w/w, of a dispersing agent, such as poloxamer 188; from 1 to 10% w/w, for example from 5 to 8% w/w, of a solubilising agent, such as polyethylene glycol; from 0.1 to 10% w/w, for example from 1 to 5% w/w, of a disintegrant, such as crospovidone; and from 0.1 to 10% w/w, for example from 1 to 5% w/w, of a flavourant, such as Novomint Freshmint 506038 TP0504.

The present invention also provides a buccal drug delivery device or a dose cartridge for use in a buccal drug delivery device loaded with a composition of the invention. A suitable device is the Pfeiffer Unitdose Powder nasal delivery device.

The particle size distribution of the compositions needs to be such that they are efficiently aerosolised from the delivery device and evenly distributed across the absorptive surface of the buccal cavity to aid absorption. Additionally, the drug also needs to rapidly dissolve when deposited in the buccal cavity. These properties are more readily achieved by smaller particles. Reducing the particle size, for example by milling, can also improve mouth feel, for example by reducing grittiness.

There are a number of methods for measuring the particle size distribution of powders, including sieve analysis, light microscopy and laser diffraction, for example using a Sympatec laser diffraction instrument (see C. Washington, Particle size analysis in pharmaceutics and other industries, Ellis Horwood, Chichester, 1992). The particle size distribution may be measured on the bulk powder or on the powder as it is emitted from the device.

The mean particle size (diameter) of the powder is preferably in the range 50 to 500 μm, more preferably in the range 50 to 200 μm and most preferably in the range 80 to 120 μm, for example about 100 μm. The percentage of particles (by volume) below 25 μm is preferably less than 25%, more preferably less than 20% and most preferably less than 15%; for example no more than 10% less than 20 μm and at least 99% less that 500 μm.

The present invention provides processes for preparing the compositions of the invention. These processes are hereinbefore described.

The compositions of the invention can be used to treat and/or prevent certain disorder conditions or diseases of the central nervous system and in particular can be used to cause sedation, hypnosis, decreased anxiety, muscle relaxation, anterograde amnesia and anticonvulsant actions. They can also be used to treat and/or prevent anxiety, epilepsy, insomnia, alcohol dependence, muscular disorders and mania. Thus, the present invention provides a method of administering a benzodiazepine drug compound, particularly a compound as listed above, to a patient in need thereof, for example for the prevention or treatment of the disorders, conditions or diseases set out above and/or to induce the effects set out above, which comprises the buccal administration of a composition as defined above to the patient.

The compositions of the present invention are particularly suitable for the treatment of epilepsy (status epilepticus). In a particular aspect, the present invention provides a composition comprising lorazepam for use in the treatment of epilepsy and a method for treating epilepsy which comprises the buccal administration of a composition of the invention to a patient.

As used herein, we use the term “patient” to refer to both human and non-human animals, e.g. mammals. The invention is particular suitable for use in the treatment of mammals, especially humans, and animals, such as, dogs.

The present invention also provides the use of a benzodiazepine drug, such as a drug as listed above and a muco-adhesive as described above in the manufacture of a medicament, e.g. a powdered medicament, for buccal administration to a patient in need of the drug. Such a medicament, e.g. a powdered medicament, may be for the treatment and/or prevention of disorders, conditions or diseases of the central nervous system and/or to induce sedation, hypnosis, decreased anxiety, muscle relaxation, anterograde amnesia and anticonvulsant actions or to treat anxiety, epilepsy, insomnia, alcohol dependence, muscular disorders or mania. In a particular aspect, the present invention provides for a use as described in this paragraph. wherein the drug is lorazepam.

The present invention also provides powder compositions comprising a benzodiazepine drug compound and a muco-adhesive as described above and optionally additional ingredients as defined above for buccal delivery for use in treating and/or prevention of disorders, conditions or diseases of the central nervous system and/or to induce sedation. Hypnosis, decreased anxiety, muscle relaxation, anterograde amnesia and anticonvulsant actions or to treat anxiety, epilepsy, insomnia, alcohol dependence, muscular disorders or mania. In a particular aspect, the present invention provides a powder composition comprising lorazepam for use in the treatment of the conditions listed in this paragraph, in particular epilepsy.

The invention is illustrated by the following non-limiting Examples.

### EXAMPLE 1

A composition having the following composition was produced.

| Component | mg/Unit | % |
|---|---|---|
| *Lorazepam EP (optionally milled) | 4.00 | 5.41 |
| Propylene Glycol Alginate (Protanal Ester SD-LB) | 20.50 | 27.70 |
| Xylitol 100 | 27.75 | 37.50 |
| Poloxamer 188 (Lutrol micro 68) | 13.00 | 17.57 |
| Polyethylene Glycol 20000 powder | 5.25 | 7.09 |
| Polyplasdone XL 10 (CrosPovidone) | 1.75 | 2.36 |
| Novomint Freshmint 506038 TP0504 | 1.75 | 2.36 |
| TOTAL | 74.00 | 100.00 |

*Note that the dose of Lorazepam can be varied—typically from 0.5 g to 10 mg

The Lorazepam was milled using a planetary ball mill to enhance absorption and eliminate any residual mouth feel grittiness. The particle size was measured using a Sympatec laser diffraction instrument. The mean particle size of the lorazepam was about 3 μm and the mean particle size of the blend was about 100 μm.

1. A powder composition for buccal delivery comprising a drug compound, a muco-adhesive agent and a dispersing agent.

2. A powder composition according to claim 1 wherein the drug compound is a benzodiazepine drug compound.

3. A powder composition according to claim 1, wherein the drug compound is selected from the group alprazolam, chlordiazepoxide, clonazepam, clorazepate, diazepam, estazolam, flurazepam, halazepam, lorazepam, midazolam, nitrazepam, oxazepam, prazepam, quazepam, temazapem, bromazepam, flunitrazepam, triazolam, bentazepam, brotizolam, clotiazepam, delorazepam, ethyl loflazepate, etizolam, fludiazepam, ketozolam, loprazolam, lormetazepam, nordazepam, mexazolam, nimetazepam, pinazepam and tetrazepam, and optionally combinations thereof.

4. A powder composition according to claim 1 wherein the drug compound is lorazepam.

5. A powder composition according to claim 1 wherein the muco-adhesive agent is selected from (i) esters and salts of alginic acid and (ii) hydroxyethyl cellulose.

6. A powder composition according to claim 1 wherein the muco-adhesive agent comprises propylene glycol alginate or sodium alginate.

7. A powder composition according to claim 1 wherein the dispersing agent is selected from ethylene oxide-propylene oxide copolymers.

8. A powder composition according to claim 7 wherein the dispersing agent comprising poloxamer 188.

9. A powder composition according to claim 1 which comprises a freeze-dried powder or comprises granules.

10. A powder composition according to claim 9 which comprises granules and the granules comprise the drug compound and the muco-adhesive agent.

11. A powder composition according to claim 10 which comprises granules and the drug compound and the muco-adhesive agent, and optionally other ingredients, are incorporated within a single granule.

12. A powder composition according to claim 1 wherein the powder composition is loaded in a buccal drug delivery device or a dose cartridge for use in said buccal drug delivery device.

13-17. (canceled)

18. A powder composition according to claim 1 comprising a benzodiazepine drug compound and a muco-adhesive agent selected from esters and salts of alginic acid and hydroxyethyl cellulose for use in buccal administration to treat and/or prevent disorders, conditions or diseases of the central nervous system.

19. A powder composition according to claim 1 comprising a benzodiazepine drug compound and a muco-adhesive agent, wherein the muco-adhesive agent is selected from esters and salts of alginic acid and hydroxyethyl cellulose, for use in buccal administration to induce sedation, hypnosis, decreased anxiety, muscle relaxation, anterograde amnesia or anticonvulsant actions.

20. A powder composition according to claim 1 comprising a benzodiazepine drug compound and a muco-adhesive agent, wherein the muco-adhesive agent is selected from esters and salts of alginic acid and hydroxyethyl cellulose for use in buccal administration to treat and/or prevent anxiety, epilepsy, insomnia, alcohol dependence, muscular disorders or mania.

21. A powder composition according to claim 1 comprising a benzodiazepine drug compound wherein the benzodiazepine drug compound is lorazepam and the condition to be treated and/or prevented is epilepsy.

22-23. (canceled)

24. A method of treating or preventing disorders, conditions or diseases of the central nervous system, which method comprises the buccal administration of a composition comprising a drug compound, a muco-adhesive agent and a dispersing agent.

25. A method of treating or preventing disorders, conditions or diseases of the central nervous system, which method comprises the buccal administration of a powder composition comprising a benzodiazepine drug and a muco-adhesive agent selected from esters and salts of alginic acid and hydroxyethyl cellulose for use in buccal administration to treat and/or prevent disorders, conditions or diseases of the central nervous system.

26. (canceled)

27. A method of inducing sedation, hypnosis, decreased anxiety, muscle relaxation, anterograde amnesia or anticonvulsant actions, which method comprises the buccal administration of a composition comprising a benzodiazepine drug and a muco-adhesive agent selected from esters and salts of alginic acid and hydroxyethyl cellulose for use in buccal administration to treat and/or prevent disorders, conditions or diseases of the central nervous system.

28. (canceled)

29. A method of treating or preventing anxiety, epilepsy, insomnia, alcohol dependence, muscular disorders or mania, which method comprises the buccal administration of a composition comprising a benzodiazepine drug and a muco-adhesive agent selected from esters and salts of alginic acid and hydroxyethyl cellulose for use in buccal administration to treat and/or prevent disorders, conditions or diseases of the central nervous system.
